(19) United States
(12) Patent Application Publication
(10) Pub. No.: US 2019/0315704 A1
(43) Pub. Date: Oct. 17, 2019

(54) PROTECTION OF PLANT EXTRACTS AND COMPOUNDS FROM DEGRADATION

(71) Applicant: Medlab IP Pty Ltd, Alexandria, New South Wales (AU)

(72) Inventors: Luis VITETTA, Varsity Lakes, Queensland (AU); Sean HALL, Millers Point, New South Wales (AU)

(73) Assignee: Medlab IP Pty Ltd, Alexandria, New South Wales (AU)

(21) Appl. No.: 16/300,537

(22) PCT Filed: May 11, 2017
(86) PCT No.: PCT/AU2017/050430

§ 371 Date: Nov. 9, 2018

(30) Foreign Application Priority Data
May 11, 2016 (AU) 2016901758

(51) Int. Cl. C07D 311/80

(52) U.S. Cl. CPC C07D 311/80

(57) ABSTRACT

Provided herein are methods for protecting cannabinoids, typically in medicinal compositions, from heat-, sunlight- or artificial-light-induced degradation, and oxidative degradation due to contact with ambient air/oxygen. Also provided are methods for extending the shelf life, stability, and long term viability and efficacy of cannabis- and cannabinoid-containing medicinal compositions. Said cannabinoids are protected by providing the one or more cannabinoids in a composition comprising at least one non-ionic surfactant and at least one polyol.

### FIELD OF THE ART

The present invention relates to methods for protecting cannabinoids, typically in medicinal compositions, from heat, sun-light- or artificial-light-induced degradation, and oxidative degradation due to contact with ambient air or oxygen. Therefore the invention also relates to methods for increasing the shelf life, stability, and long term viability and efficacy of cannabis- and cannabinoid-containing medicinal compositions.

### BACKGROUND

Cannabis has been utilised for centuries throughout the world to alleviate disease. Its derivatives were named a panacea, or as a cure-all, and were sold as a legal medicine, mainly for pain. Currently available treatments, generally opioids and anti-inflammatory drugs, are not always effective for certain painful conditions.

Cannabinoids have anti-nociceptive mechanisms different from those of other drugs currently in use, which thus opens a new line of potential treatment to mitigate pain that is non-responsive to current pharmacologic treatments, such as opioid medications, especially for neuropathic and inflammation associated pain. Nevertheless, the potential for cannabis abuse is a major risk and restriction in the field.

Moreover, it has long been recognised that one of the greatest hindrances to the development of cannabis-based medicines is the effect of exposure to light on stability of the cannabis extract. Specifically cannabinoids are subject to significant oxidative degradation when in solution and when exposed to sun-light or artificial light; air/oxygen or when heated. All these exposures lead to significant losses of cannabinoid viability. Cannabinoids can decompose when exposed to air, light and heat, or exposure to an acid environment, oxidising the compound to a much less potent cannabinoid, such as in the conversion of active Δ9-tetrahydrocannabinol to the significantly less active cannabinol.

There is a need for the development of methods for stabilising compositions comprising cannabinoids and protecting such compositions from oxidation and degradation over time, to thereby increase the shelf life and preserve the long term viability and efficacy of cannabis- and cannabinoid-based medicines.

### SUMMARY OF THE DISCLOSURE

The present inventors have now surprisingly found that a micellised formulation of cannabis oil extract, formulated as described herein, protects cannabinoids from oxidation and degradation over time, when exposed to sunlight or artificial light, heat or due to contact with ambient air/oxygen. Thus, the present disclosure provides cannabis and cannabinoid-containing compositions with increased shelf-life compared to compositions of the prior art.

A first aspect of the disclosure provides a method for protecting one or more cannabinoids from degradation, the method comprising providing the one or more cannabinoids in a composition comprising at least one non-ionic surfactant and at least one polyol.

The degradation may comprise light-induced degradation, heat-induced degradation or degradation due to contact with ambient air or oxygen.

A further aspect of the disclosure provides a method for protecting one or more cannabinoids from oxidation, the method comprising providing the one or more cannabinoids in a composition comprising at least one non-ionic surfactant and at least one polyol.

A further aspect of the disclosure provides a method for extending the shelf life or stability of one or more cannabinoids in solution, by providing the cannabis or cannabinoids in a composition comprising at least one non-ionic surfactant and at least one polyol.

In accordance with the method, the shelf life may be shelf life at or above room temperature. The stability may be stability at or above room temperature. The shelf life or stability of the one or more cannabinoids in solution may be more than 26 weeks, or more than 52 weeks.

A further aspect of the disclosure provides a method for extending the room temperature stability or shelf life of cannabis- and cannabinoid-containing medicaments, by providing the cannabis or cannabinoids in a composition comprising at least one non-ionic surfactant and at least one polyol.

In accordance with the method, the stability or shelf life of the medicaments may be extended to more than 26 weeks, or more than 52 weeks.

In accordance with the present disclosure, the one or more cannabinoids may be present in the composition in the form of a cannabis plant extract or hemp seed oil.

In accordance with the present disclosure, the composition may further comprise at least one oil. The at least one oil may be ethyl oleate, ethyl linoleate, caproic acid, caprylic acid, capric acid, or lauric acid, or a combination thereof. The at least one oil may be a natural oil or be derived from a natural oil. The natural oil may be coconut oil, palm kernel oil, palm oil, lemon oil, or sunflower oil, or a combination thereof.

In accordance with the present disclosure, the at least one non-ionic surfactant may be selected from the group consisting of a polyethoxylated castor oil, polyoxyethylene sorbitan monolaurate, polyoxyethylene sorbitan monoleate, tocopheryl polyethylene glycol succinate and mixtures thereof. The at least one non-ionic surfactant may be a polyethoxylated castor oil. The at least one non-ionic surfactant may be obtained by reacting castor oil or hydrogenated castor oil with ethylene oxide. The at least one non-ionic surfactant may comprise polyethylene glycol ricinoleate, fatty acid esters of polyethylene glycol, free polyethylene glycols and ethoxylated glycerol. The at least one non-ionic surfactant may comprise polyethylene glycol hydroxystearate, fatty acid glycerol polyglycol esters, polyethylene glycols and glycerol ethoxylate.

In accordance with the present disclosure, the at least one polyol may be selected from the group consisting of glycerol and propylene glycol.

In accordance with the present disclosure, the composition may further comprise a non-aqueous solvent or a mixture of a non-aqueous solvent and water.

In accordance with the present disclosure, the at least one non-ionic surfactant may have an Hydrophile-Lipophile Balance (HLB) value of from about 8 to about 20. The at least one non-ionic surfactant may have an Hydrophile-Lipophile Balance (HLB) value of from about 12 to about 16.

In accordance with the present disclosure, the composition may have a viscosity of from about 5 cP to about 35 cP.

In accordance with the present disclosure, the composition may have an average particle size of from about 5 nm to about 200 nm.

In accordance with the present disclosure, the ratio of the at least one non-ionic surfactant to the at least one polyol in the composition may be from about 2:1 to about 1.5:1 by weight.

A further aspect of the disclosure provides a shelf stable cannabis- or cannabinoid-containing medicament or formulation, wherein a cannabis extract or one or more cannabinoids are provided in a composition comprising at least one non-ionic surfactant and at least one polyol, and wherein the shelf stability of the medicament or formulation is greater than 1 week, greater than 5 weeks, greater than 10 weeks, greater than 20 weeks, greater than 30 weeks, greater than 40 weeks, greater than 50 weeks, or greater than 52 weeks at or above room temperature.

The shelf stable medicament or formulation may be kept, at least for some period of time, in lighted conditions. The shelf stable medicament or formulation may be kept in conditions under which it is exposed, at least for some period of time, to ambient air or oxygen. The shelf stable medicament or formulation may be kept in conditions where it is exposed, at least for some period of time, to temperatures above room temperature.

### BRIEF DESCRIPTION OF THE DRAWINGS

Embodiments of the present disclosure are described herein, by way of non-limiting example only, with reference to the following drawings.

FIG. 1. Demonstration of stability (protection from oxidative damage or degradation) of cannabinoids in a composition of the present disclosure compared to the degradation of cannabis plant material and of cannabinoids in a prior art cannabis oil extract. Stability of the exemplified composition according to the present disclosure is represented by the maintenance of the uniform appearance of the liquid composition ands the absence of separation into distinct phases. A, Less than five days after preparation of the exemplified composition of the present disclosure (right hand tube), compared to cannabis plant material (left hand tube) and of cannabinoids in a prior art cannabis oil extract (middle tube). B, As for A, after 6 weeks at room temperature. C, As for A, after 15 weeks at room temperature. D, After 52 weeks at room temperature; exemplified composition of the present disclosure (right hand tube), compared to cannabinoids in a prior art cannabis oil extract (left hand tube).

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

Throughout this specification and the claims which follow, unless the context requires otherwise, the word “comprise”, and variations such as “comprises” or “comprising”, will be understood to imply the inclusion of a stated element, integer or step, or group of elements, integers or steps, but not the exclusion of any other element, integer or step, or group of elements, integers or steps.

The articles “a” and “an” are used herein to refer to one or to more than one (i.e., to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

As used herein, the term “room temperature” refers to a temperature in the range of about 15° C. to about 25° C. In particular embodiments, room temperature may refer to a temperature of between about 20° C. and about 22° C.

Terpenes are a class of organic compound, where the structure of each member is derived from multiple units of isoprene, a hydrocarbon motif containing alternating single and double bonds. Where the terpene contains a non-hydrocarbon functional group, such as oxygenated terpene derivative, a terpenoid is formed. Both terpenes and terpenoids are typically naturally occurring and are found in a variety of plants and their flowers, their presence often being responsible for the characteristic odour of these plants. Terpenes and terpenoids are also found as principal components of essential oils, which are often derived from plants.

The cannabis plant comprises a large proportion of terpenes and terpenoids, which are largely responsible for the characteristic odour of the plant. The extraction of cannabinoids such as THC from the cannabis plant typically resulting in the concurrent isolation of the terpenes and terpenoids.

Like many other naturally occurring molecules, terpenes and terpenoids can be susceptible to oxidation. As terpenes and terpenoids are derived from isoprene, their structures may also have multiple double bonds, which may increase their susceptibility to oxidation. Where oxidation of a terpene or terpenoid occurs, this may result in the formation of new double bonds and rearrangement of the molecule, leading to a part of or the whole of the molecule adopting a conjugated arrangement. Sufficient conjugation then leads to the molecule being capable of absorbing light in the visible spectrum, which results in the compound being “coloured”. Oxidation of terpenes and terpinoids to produce conjugated analogues is often observed through discolouration of the compounds, the detection of which can be used as an indication of the extent of oxidation.

The visible oxidation of terpenes and terpinoids in cannabis extracts and solutions is an indicator of loss of stability (e.g. oxidation) of cannabinoids. Thus, the extent of oxidation in compositions comprising cannabinoids can be determined visually (by reference to terpene or terpinoid-induced discolouration of the composition). Alternatively, oxidation can also be measured by a variety of analytical techniques well known to those skilled in the art such as, but not limited to, gas chromatography mass spectrometry and related techniques.

As described herein and in international (PCT) patent application no. PCT/US2016/020468 (the disclosure of which is incorporated herein by reference), the inventors have developed a formulation for delivery, optionally transmucosal and transdermal delivery, of cannabinoids, comprising nano-sized particles with a inner hydrophobic core of cannabis oil extract combined with lipid carrier and a hydrophilic outer shell creating a water soluble solution that easily crosses the mucosal cell physical barrier.

The present inventors have now surprisingly discovered that this formulation environment protects the cannabinoids from the oxidation and degradation that otherwise occurs in cannabis oil extracts in solution. Thus, the long term stability at room temperature, and hence shelf-life of cannabis- and cannabinoid-containing compositions can be increased beyond that which would have been predicted possible.

Accordingly, provided herein are methods for protecting one or more cannabinoids from degradation or oxidation, the method comprising providing the one or more cannabinoids in a composition comprising at least one non-ionic surfactant and at least one polyol.

Also provided are methods for extending the room temperature stability or shelf life of one or more cannabinoids in solution, and of cannabis- and cannabinoid-containing medicaments and formulations, by providing the cannabis or cannabinoids in a composition comprising at least one non-ionic surfactant and at least one polyol.

The shelf life and/or stability of cannabinoids and cannabis extracts may be extended, in accordance with the present disclosure, beyond that achievable in the absence of providing the cannabinoids and cannabis extracts in a composition comprising at least one non-ionic surfactant and at least one polyol. For example, shelf life and/or stability may be extended beyond 1 week, beyond 5 weeks, beyond 10 weeks, beyond 20 weeks, beyond 30 weeks, beyond 40 weeks, beyond 50 weeks, or beyond 52 weeks.

The extended shelf life and/or stability may be achieved for solutions, medicaments and formulations kept, at least for some period of time, in lighted conditions, under conditions under which they are exposed, at least for some period of time, to ambient air or oxygen, and/or under conditions where they are exposed, at least for some period of time, to temperatures above room temperature. By way of example, the temperature may reach about 25° C., about 30° C., about 35° C., about 40° C., about 45° C., about 50° C., about 55° C., about 60° C., about 65° C. or about 70° C.

Accordingly, also provided herein are shelf stable cannabis- and cannabinoid-containing medicaments, wherein a cannabis extract or one or more cannabinoids are provided in a composition comprising at least one non-ionic surfactant and at least one polyol, and wherein the shelf stability of the medicaments is greater than 1 week, greater than 5 weeks, greater than 10 weeks, greater than 20 weeks, greater than 30 weeks, greater than 40 weeks, greater than 50 weeks, or greater than 52 weeks at or above room temperature.

The shelf stable medicament or formulation may be kept, at least for some period of time, in lighted conditions. The shelf stable medicament or formulation may be kept in conditions under which it is exposed, at least for some period of time, to ambient air or oxygen. The shelf stable medicament or formulation may be kept in conditions where it is exposed, at least for some period of time, to temperatures above room temperature. By way of example, the temperature may reach about 25° C., about 30° C., about 35° C., about 40° C., about 45° C., about 50° C., about 55° C., about 60° C., about 65° C. or about 70° C.

The one or more cannabinoids may be present in the composition in the form of, for example, a cannabis plant extract or hemp seed oil. As used herein the term “extract” refers to an active preparation derived from one or more plants or a synthetic version thereof. In the context of the specification by “active” it is meant that the extract is capable of producing a desired therapeutic benefit. An extract is obtained by a process of “extraction” which will be understood by those skilled in the art as, in general terms, comprising treating plant material with a solvent, a liquid, or a supercritical fluid to dissolve the active preparation and separate the same from residual unwanted plant material. An extract may be in liquid form (for example as a decoction, solution, infusion, oil or tincture) or solid form (for example as a powder or granules). An extract may comprise a single active agent or a combination of active agents.

The term “cannabinoid” as used herein encompasses natural cannabinoids, natural cannabinoids that have been purified or modified, and synthetically derived cannabinoids. Exemplary cannabinoids include, but are not limited to, delta-8-tetrahydrocannabinol, delta-9-tetrahydrocannabinol, cannabidol, olivetol, cannabinol, cannabigerol, nabilone, delta-9-tetrahydro cannabinotic acid, 3-dimethylnepty 11 carboxylic acid homologine 8, delta-8-tetrahydrocannabinol, prodrugs of cannabinoids, as well as pharmaceutically acceptable salts and complexes thereof.

Described herein are compositions and delivery systems comprising at least one non-ionic surfactant, at least one polyol, and one or more cannabinoids. In some embodiments the composition or delivery system further comprises at least one oil. In some embodiments, the present invention beneficially provides delivery of one or more cannabinoids in a stable, protected composition or delivery system via the oral and/or nasal transmucosal or transdermal route, thereby bypassing the gastrointestinal tract and avoiding the need to break the skin, as with, for example an injection, and ensuring rapid absorption by the bloodstream.

As used herein the term “delivery system” refers to a composition comprising a formulation according to the invention which is particularly adapted for delivery of active agents transdermally, orally or via the oral and/or nasal mucosa. Typically the particle size of the delivery system is from about 1 to about 200 nm. The terms “composition” and “delivery system” may be used interchangeably herein.

As used herein, the term “oral” refers to a composition or delivery system that can be administered orally. The term “oral” includes ingestion and oral transmucosal delivery, for example, buccal and/or sublingual delivery.

As used here, the term “transdermal” refers to a composition or delivery system that can be administered to the skin, wherein the active agent is delivered across the skin for systemic distribution.

Transmucosal delivery systems according to the invention may be in any form suitable for delivery of active agents via the oral and/or nasal mucosa, including for example, but not limited to, sprays, pumps, gels including mucoadhesive polymeric gels, foams and quick dissolve tablets. The skilled artisan will appreciate that the transmucosal delivery systems are not so limited and that any transmucosal formulations may be employed.

Transdermal delivery systems according to the invention may be in any form suitable for delivery of active agents transdermally, including for example, but not limited to, sprays, lotions, gels, creams, patches and implants. The transdermal delivery systems may employ chemical enhancers, which aid dermal penetration and/or use ultrasound or iontophoresis for the improved delivery of drug molecules that do not easily undergo passive diffusion. Microneedles and electroporation type transdermal delivery forms may also be used with the delivery systems according to the invention. The skilled artisan will appreciate that the transdermal delivery systems are not so limited and that any transdermal formulations may be employed.

In particular embodiments the formulation of the delivery system is such that it provides a fine micellized mist spray comprising the active agent, the spray being suitable for administration orally, for buccal or other oral mucosal delivery, intranasally for delivery via the nasal mucosa, or transdermally across the skin. Without wishing to be bound by theory, the fine mist ensures maximum surface coverage and therefore optimum delivery of the active agent(s) via the oral and/or nasal mucosa. In some embodiments the Hydrophile-Lipophile Balance (HLB) of the non-ionic surfactant, the particular ratio of the at least one active agent to the at least one non-ionic surfactant, and the ratio of the at least one non-ionic surfactant to the at least one polyol assist to provide a delivery system with the necessary characteristics such as small particle size and viscosity, to produce a formulation adapted for oral and/or nasal transmucosal delivery.

While a fine mist spray for oral delivery via the buccal mucosa, intranasal delivery via the nasal mucosa, or transdermal delivery across the skin is one particularly advantageous form of the delivery system, those skilled in the art will appreciate that the delivery system may be delivered in a form other than a spray. Sprays and other forms of the delivery systems of the invention may be administered using any suitable conventional administration means. For example a spray delivery system of the invention may be administered via a pump action or pressurized administration vessel such as an aerosol spray. In particular embodiments the administration means may provide metered doses of the composition. In some embodiments the delivery systems are absorbed onto solid carriers such as, but not limited to, powders, granules, or beads. The powders may include lyophilised bacteria.

In accordance with embodiments of the invention, the delivery systems may be administered transdermally to any external skin of a subject. The skin area may be, for example, the scalp, hands, arms, underarms, face, groin or feet. Those skilled in the art will readily appreciate however that any surface, organic or inorganic in nature, may be the subject of treatment in accordance with embodiments of the invention. Transdermal delivery systems of the invention may further comprise one or more preservatives, moisturizers, carriers, excipients, diluents and/or adjuvants. In a particular embodiment the transdermal delivery system comprises phenoxyethanol as a preservative and allantoin as a moisturizer. In particular embodiments the pH of the transdermal delivery system may be adjusted to about pH 4 to about pH 8. Typically, the pH of the transdermal delivery system is from about pH 5.0 to pH 6.0. For example, the pH of the transdermal delivery system may be about pH 5.1, pH 5.2, pH 5.3, pH 5.4, pH 5.5, pH 5.6, pH, 5.7, pH 5.8, or pH 5.9.

Transdermal delivery systems of the invention may also include compounds which enhance dermal penetration, such as, for example, anionic surfactants, cationic surfactants, zwitterionic surfactants, nonionic surfactants, fatty acids, fatty esters, fatty amines, terpenes, sulphoxides, laurocapram, pyrrolidones, alcohol, glycol, urea and skin penetration enhancing peptides.

The compositions or delivery systems may further comprise at least one oil. In some embodiments the one or more cannabinoids, cannabis extract or hemp seed oil may be provided in the oil or in an oil/solvent mixture. The oil or oil mixture may act as a carrier or solvent for the one or more cannabinoids. The oil or oil mixture may increase the stability of the composition or delivery system. The oil may be a natural oil in that it is animal, plant or petrochemical in origin; may be derived from or extracted from a natural oil via a physical or chemical process; or may be synthetic oil. Examples of suitable oils include, but are not limited to lemon oil, sunflower oil, soybean oil, canola oil, olive oil, corn oil, peanut oil, groundnut oil, rice bran oil, coconut oil, cottonseed oil, flax seed oil, palm oil, palm kernel oil, safflower oil, soybean oil, sesame oil, amaranth oil, linseed oil, argan oil, grapeseed oil, cranberry seed oil. hazelnut oil. hemp oil, jojoba oil, macadamia oil, mustard oil, neem oil, orange oil, rapeseed oil, avocado oil, almond oil, sweet almond oil, cashew oil, castor oil, vegetable oil, walnut oil, wheatgerm oil, kukui nut oil, tamuna oil, aloe vera oil, apricot kernel oil, borage oil (from, for example Borago officionalis), camellia oil (from, for example, Camellia oleifera), cocoa butter oil, rosehip see oil, fish oils, ethyl oleate, ethyl linoleate, saturated fatty acids (such as, but not limited to, caproic acid, caprylic acid, capric acid, lauric acid, valeric acid, myristic acid, palmitic acid, stearic acid, arachidic acid), medium chain triglycerides, omega-3 fatty acids (such as, but not limited to, hexadecatrienoic acid, alpha-linolenic acid, stearidonic acid, ecosatrienoic acid, eicosapentaenoic acid, heneicosapentanoic acid, docosapentanoic acid, docosahexanoic acid, tetracosapentaenoic acid, tetracosahexanenoic acid), omega-6 fatty acids (such as, but not limited to, linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, tetracosapentaenoic acid), and/or omega-9 fatty acids (such as, but not limited to, oleic acid, eicosenoic acid, mead acid, erucic acid, nervonic acid). In particular embodiments the oil is olive oil, medium chain triglycerides, ethyl oleate, ethyl linoleate, caproic acid, caprylic acid, capric acid, or lauric acid, or a combination thereof. The oil mixture may comprise an oil, a non-aqueous solvent (such as an organic solvent or an inorganic solvent and/or mixtures thereof) and/or water. The oil may be a natural oil in that it is animal, plant or petrochemical in origin; may be derived from or extracted from a natural oil via a physical or chemical process; or may be synthetic oil. Suitable organic solvents are known to those skilled in the art and may include, but are not limited to polar solvents (for example ethanol), non-polar solvents (for example hexane) and/or halogenated solvents (for example dichloromethane).

The ratio of oil and one or more cannabinoids, cannabis extract or hemp seed oil to the at least one non-ionic surfactant may be from about 1:2 to about 1:8 by weight. Typically, the ratio of oil and one or more cannabinoids, cannabis extract or hemp seed oil to the at least one non-ionic surfactant may be from about 1:4.5 to about 1:6.5 by weight. For example, the ratio of oil and the oil and one or more cannabinoids, cannabis extract or hemp seed oil to the at least one non-ionic surfactant may be from to the at least one non-ionic surfactant may be about 1:4.6 by weight, 1:4.7 by weight, 1:4.8 by weight, 1:4.9 by weight, 1:5.0 by weight, 1:5.1 by weight, 1:5.2 by weight, 1:5.3 by weight, 1:5.4 by weight, 1:5.5 by weight, 1:5.6 by weight, 1:5.7 by weight, 1:5.8 by weight, 1:5.9 by weight, 1:6.0 by weight, 1:6.1 by weight, 1:6.2 by weight, 1:6.3 by weight, or 1:6.4 by weight.

In some embodiments the non-ionic surfactant may have an Hydrophile-Lipophile Balance (HLB) value of from about 8 to about 20. In accordance with particular embodiments of the invention the non-ionic surfactant may have an Hydrophile-Lipophile Balance (HLB) value of from 10 to 18, or more typically of from 11 to 17. For example, the non-ionic surfactant may have an Hydrophile-Lipophile Balance (HLB) value of 11, 12, 13, 14, 15, 16 or 17.

In some embodiments the viscosity of the delivery system may be about 5 cP to about 35 cP. In accordance with particular embodiments of the invention the viscosity of the delivery system is from about 8 cP to about 30 cP. For example, the viscosity of the delivery system may be about 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP or 30 cP.

In some embodiments the average size of particles in the delivery system may be up to about 300 nm. In accordance with particular embodiments of the invention the average particle is from about 1 nm to about 250 nm. In accordance with particular embodiments of the invention the average particle size is about 5 nm, 10 nm, 15 nm, 20 nm, 25 nm, 30 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, or 200 nm. In accordance with particular embodiments of the invention the average particle size is from about 1 nm to about 100 nm. For example, the particle size may be about 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm 94 nm, 95 nm, 96 nm, 97 nm, 98 nm, 99 nm or 100 nm.

In some embodiments the ratio of the one or more cannabinoids, cannabis extract or hemp seed oil to the at least one non-ionic surfactant may be from about 1:5 to about 1:15 by weight. For example, the ratio of the one or more cannabinoids, cannabis extract or hemp seed oil to the at least one non-ionic surfactant may be about 1:5 by weight, 1:5.5 by weight, 1:6 by weight, 1:6.5 by weight, 1:7 by weight, 1:7.5 by weight, 1:8 by weight, 1:8.5 by weight, 1:9 by weight, 1:9.5 by weight, 1:10 by weight, 1:10.5 by weight, 1:11 by weight, 1:11.5 by weight, 1:12 by weight, 1:12.5 by weight, 1:13 by weight, 1:13.5 by weight, 1:14 by weight, 1:14.5 by weight or 1:15 by weight.

In some embodiments the ratio of the at least one non-ionic surfactant to the at least one polyol may be from about 2:1 to about 1.5:1 by weight. For example, the ratio of the at least one non-ionic surfactant to the at least one polyol may be about 2:1 by weight, 1.99:1 by weight, 1.98:1 by weight, 1.97 by weight, 1.96 by weight, 1.95:1 by weight, 1.94:1 by weight, 1.93:1 by weight, 1.92:1 by weight, 1.91:1 by weight, 1.90:1 by weight, 1.89:1 by weight, 1.88:1 by weight, 1.87 by weight, 1.86 by weight, 1.85:1 by weight, 1.84:1 by weight, 1.83:1 by weight, 1.82:1 by weight, 1.81:1 by weight, 1.80:1 by weight, 1.79:1 by weight, 1.78:1 by weight, 1.77 by weight, 1.76 by weight, 1.75:1 by weight, 1.74:1 by weight, 1.73:1 by weight, 1.72:1 by weight, 1.71:1 by weight, 1.70:1 by weight, 1.69:1 by weight, 1.68:1 by weight, 1.67 by weight, 1.66 by weight, 1.65:1 by weight, 1.64:1 by weight, 1.63:1 by weight, 1.62:1 by weight, 1.61:1 by weight, 1.60:1 by weight, 1.59:1 by weight, 1.58:1 by weight, 1.57 by weight, 1.56 by weight, 1.55:1 by weight, 1.54:1 by weight, 1.53:1 by weight, 1.52:1 by weight, 1.51:1 by weight or 1.5:1 by weight.

In some embodiments, the composition or delivery system further comprises water. The ratio of water to the one or more cannabinoids, cannabis extract or hemp seed oil, at least one non-ionic surfactant and at least one polyol may be from about 4:1 to about 1:1 by weight. Typically, the ratio of water to the one or more cannabinoids, cannabis extract or hemp seed oil, at least one non-ionic surfactant and at least one polyol is from about 3.5:1 to about 2:1 by weight. For example, the ratio of water to the one or more cannabinoids, cannabis extract or hemp seed oil, at least one non-ionic surfactant and at least one polyol may be about 3.5:1 by weight, 3.4:1 by weight, 3.3:1 by weight, 3.2:1 by weight, 3.1:1 by weight, 3.0:1 by weight, 2.9:1 by weight, 2.8:1 by weight, 2.7:1 by weight, 2.6:1 by weight, 2.5:1 by weight, 2.4:1 by weight, 2.3:1 by weight, 2.2:1 by weight, 2.1:1 by weight or 2:1 by weight.

The compositions and delivery systems of the invention comprise at least one non-ionic surfactant. The at least one non-ionic surfactant may include, but is not limited to, one or more of a polyethoxylated castor oil, polyoxyethylene sorbitan monolaurate, polyoxyethylene sorbitan monoleate and tocopheryl polyethylene glycol succinate, however a person skilled in the art will appreciate that other non-ionic surfactants may also be used. The at least one non-ionic surfactant may obtained by reacting castor oil or hydrogenated castor oil with ethylene oxide. In certain embodiments the at least one non-ionic surfactant comprises polyethylene glycol ricinoleate, glycerol polyethylene glycol ricinoleate, fatty acid esters of polyethylene glycol, free polyethylene glycols and ethoxylated glycerol. In other embodiments the at least one non-ionic surfactant comprises polyethylene glycol hydroxystearate, glycerol polyethylene glycol hydroxystearate, fatty acid glycerol polyglycol esters, polyethylene glycols and glycerol ethoxylate. In one embodiment the at least one non-ionic surfactant is a polyethoxylated castor oil.

The compositions and delivery systems of the invention comprise at least one polyol. The at least one polyol may include, but is not limited to, glycerol or propylene glycol, however a person skilled in the art will appreciate that other polyols may also be used. In one embodiment the at least one polyol is glycerol.

In further embodiments the compositions and delivery systems may further comprise at least one flavour. In particular embodiments the flavour is a natural oil. Typically, the natural oil is peppermint oil or orange oil, however a person skilled in the art will appreciate that other natural oils may also be used to impart a pleasant flavour and/or aroma to the delivery systems.

In further embodiments the compositions and delivery systems may further comprise at least one acidulant. Typically the at least one acidulant is citric acid, acetic acid or lactic acid, however a person skilled in the art will appreciate that other acidulants may also be used to modify and/or maintain the pH of the delivery systems. In a particular embodiment the acidulate is citric acid.

In further embodiments the compositions and delivery systems may further comprise at least one preservative. Typically the at least one preservative is potassium sorbate or phenoxyethanol, however a person skilled in the art will appreciate that other preservatives may also be used.

In further embodiments the compositions and delivery system may further comprise at least one sweetener. The at least one sweetener is typically, but not limited to, a natural sweetener. The natural sweetener may be stevia, erythritol, xylitol, mannitol and/or sorbitol, however a person skilled in the art will appreciate that other natural sweeteners may also be used. In particular embodiments the natural sweetener is stevia.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1—Cannabinoid Transmucosal Spray Delivery System

10.35 mg cannabis oil (3.0026% by weight) was added to 77.00 mg Kolliphor (EL) heated to 70° C. 32.00 mg heated glycerol was added. An aqueous solution containing 0.40 mg citric acid, 2.44 mg phenoxyethanol and 1.75 mg allantoin dissolved in 73.587 g water was then added. The resulting clear micelle composition had a specific gravity of 1.149 g/mL and a pH of about 5.5.

### Example 2—Industrial Preparation of Cannabinoid Transmucosal Spray Delivery System

The cannabis oil transdermal spray of Example 1 was prepared as a large manufacturing scale batch as follows:
1. In a suitable jacketed vessel Kolliphor EL (polyethoxylated castor oil) was heated to ˜70° C.
2. Cannabis oil was added and stirred until clear.
3. Glycerine heated to ˜70° C. was added and stirred until homogeneous.
4. In a separate heating vessel water was heated to ˜70° C.
5. Phenoxyethanol was added to the heated water and stirred until completely dissolved.
6. Allantoin was added to the heated water and stirred until completely dissolved.
7. Citric acid was added to the heated water and stirred until completely dissolved.
8. The heated water solution was slowly added to the Kolliphor EL, hemp seed oil and glycerine mixture. The resulting solution was slightly cloudy.
9. The resulting solution was allowed to cool while stirring. The solution became clear as it approached room temperature.
10. The resulting pH was ˜5.5.

### Example 3—Oxidative Protection of Cannabinoids

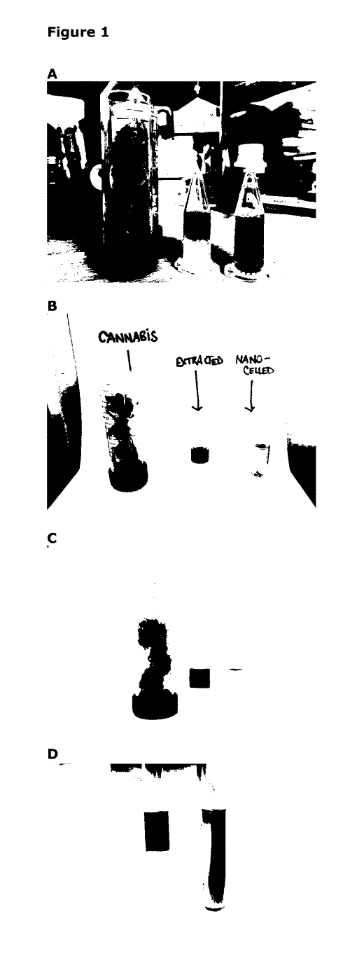

FIG. 1 illustrates the ability of a cannabis oil transmucosal/transdermal delivery system prepared as described herein to protect the cannabinoid constituents from oxidation.

Over the course of 52 weeks, at ambient temperature under light conditions, cannabis plant material substantially decomposed. Under the same conditions, a cannabis oil extract produced according to prior art methods (not prepared as described herein or in PCT/US2016/020468) developed a noticeable dark brown colour due to terpene/terpinoid oxidation. This became evident visually after only 6 weeks (FIG. 1B, middle tube) and increased over time (15 weeks, FIG. 1C (middle tube); 52 weeks, FIG. 1D (left hand tube)). In contrast, a composition comprising whole plant cannabis extract produced as described herein showed a stable dispersion with little or no evidence of oxidation over the 52 week period.

1. A method for protecting one or more cannabinoids from degradation, the method comprising providing the one or more cannabinoids in a composition comprising at least one non-ionic surfactant and at least one polyol.

2. A method according to claim 1, wherein the degradation is light-induced degradation, heat-induced degradation or degradation due to contact with ambient air or oxygen.

3. A method for protecting one or more cannabinoids from oxidation, the method comprising providing the one or more cannabinoids in a composition comprising at least one non-ionic surfactant and at least one polyol.

4. A method for extending the shelf life or stability of one or more cannabinoids in solution, the method comprising providing the one or more cannabinoids in a composition comprising at least one non-ionic surfactant and at least one polyol.

5. A method according to claim 4, wherein the shelf life is shelf life at or above room temperature.

6. A method according to claim 4, wherein the stability is stability at or above room temperature.

7. A method according to any one of claims 4 to 6, wherein the shelf life and/or stability is extended to about or beyond 52 weeks.

8. A method according to any one of claims 4 to 7, wherein the composition is stored in lighted conditions, under conditions in which it is exposed to ambient air or oxygen, or under conditions in which it is exposed to temperatures above room temperature.

9. A method according to any one of claims 1 to 8, wherein the one or more cannabinoids are present in the composition in the form of a cannabis plant extract or hemp seed oil.

10. A method according to any one of claims 1 to 9, wherein the composition further comprises at least one oil.

11. The method of claim 10, wherein the at least one oil is ethyl oleate, ethyl linoleate, caproic acid, caprylic acid, capric acid, or lauric acid, or a combination thereof.

12. The method of claim 10, wherein the at least one oil is a natural oil or is derived from a natural oil.

13. The method of claim 12, wherein the natural oil is coconut oil, palm kernel oil, palm oil, lemon oil, or sunflower oil, or a combination thereof.

14. The method of any one of claims 1 to 13, wherein the at least one non-ionic surfactant is selected from the group consisting of a polyethoxylated castor oil, polyoxyethylene sorbitan monolaurate, polyoxyethylene sorbitan monoleate, tocopheryl polyethylene glycol succinate and mixtures thereof.

15. The method of any one of claims 1 to 14, wherein the at least one non-ionic surfactant is a polyethoxylated castor oil.

16. The method of any one of claims 1 to 15, wherein the at least one non-ionic surfactant is obtained by reacting castor oil or hydrogenated castor oil with ethylene oxide.

17. The method of any one of claims 1 to 13, wherein the at least one non-ionic surfactant comprises polyethylene glycol ricinoleate, fatty acid esters of polyethylene glycol, free polyethylene glycols and ethoxylated glycerol.

18. The method of any one of claims 1 to 13, wherein the at least one non-ionic surfactant comprises polyethylene glycol hydroxystearate, fatty acid glycerol polyglycol esters, polyethylene glycols and glycerol ethoxylate.

19. The method of any one of claims 1 to 18, wherein the at least one polyol is selected from the group consisting of glycerol and propylene glycol.

20. The method of any one of claims 1 to 18, wherein the at least one polyol is glycerol.

21. The method of any one of claims 1 to 18, wherein the at least one polyol is propylene glycol.

22. The method of any one of claims 1 to 21, wherein the composition further comprises a non-aqueous solvent or a mixture of a non-aqueous solvent and water.

23. The method of any one of claims 1 to 21, wherein the composition further comprises ethanol.

24. The method of any one of claims 1 to 21, wherein the composition further comprises water.

25. The method of any one of claims 1 to 24, wherein the at least one non-ionic surfactant has an Hydrophile-Lipophile Balance (HLB) value of from about 8 to about 20.

26. The method of of any one of claims 1 to 24, wherein the at least one non-ionic surfactant has an Hydrophile-Lipophile Balance (HLB) value of from about 12 to about 16.

27. The method of any one of claims 1 to 26, wherein the composition has a viscosity of from about 5 cP to about 35 cP.

28. The method of any one of claims 1 to 27, wherein the composition has an average particle size of from about 5 nm to about 200 nm.

29. The method of any one of claims 1 to 28, wherein the ratio of the at least one non-ionic surfactant to the at least one polyol in the composition is from about 2:1 to about 1.5:1 by weight.

30. A method for extending the shelf life of a cannabis- or cannabinoid-containing medicament, comprising providing the cannabis or cannabinoids in a composition comprising at least one non-ionic surfactant and at least one polyol.

31. A shelf stable cannabis- or cannabinoid-containing medicament or formulation, wherein a cannabis extract or one or more cannabinoids are provided in a composition comprising at least one non-ionic surfactant and at least one polyol, and wherein the shelf stability of the medicament or formulation is greater than 1 week, greater than 5 weeks, greater than 10 weeks, greater than 20 weeks, greater than 30 weeks, greater than 40 weeks, greater than 50 weeks, or greater than 52 weeks at or above room temperature.

32. A shelf stable cannabis- or cannabinoid-containing medicament or formulation according to claim 31, wherein the medicament or formulation is stored in lighted conditions, under conditions in which it is exposed to ambient air or oxygen, or under conditions in which it is exposed to temperatures above room temperature.
